(19) United States
(12) Patent Application Publication
(10) Pub. No.: US 2024/0220094 A1
(43) Pub. Date: Jul. 4, 2024

(54) SYSTEMS AND METHODS INCORPORATING A LIGHT THERAPY USER INTERFACE FOR OPTICAL MODULATION

(71) Applicant: Boston Scientific Neuromodulation Corporation, Valencia, CA (US)

(72) Inventors: Joshua Dale Howard, Sacramento, CA (US); Michael A. Moffitt, Solon, OH (US); Changfang Zhu, Valencia, CA (US)

(21) Appl. No.: 18/397,740

(22) Filed: Dec. 27, 2023

Related U.S. Application Data
(60) Provisional application No. 63/437,050, filed on Jan. 4, 2023.

(51) Int. Cl. G06F 3/04847

(52) U.S. Cl. CPC G06F 3/04847; A61N 5/0601

(57) ABSTRACT

An optical modulation system includes a user interface including a plurality of user-selectable controls to select or adjust values for each of a plurality of modulation parameters and estimating a modulation dosage or irradiance at a target based on, at least, the target, a location of an optical modulation lead, values of the modulation parameters, and estimated light attenuation by tissue between the optical modulation lead and the target. An optical modulation system can limit selection of the value of at least one of the modulation parameters by at least one of a) an estimation of a modulation dosage at the target or b) an estimation of a dosage, temperature change, temperature, heating, or delivering energy at non-target issue.

### CROSS-REFERENCE TO RELATED APPLICATIONS

This application claims the benefit under 35 U.S.C. § 119(e) of U.S. Provisional Patent Application Ser. No. 63/437,050, filed Jan. 4, 2023, which is incorporated herein by reference.

### FIELD

The present invention is directed to the area of implantable optical modulation systems and methods of making and using the systems. The present invention is also directed to systems and methods for visualizing and controlling optical modulation using optical modulation leads and systems, as well as user interfaces for optical modulation systems.

### BACKGROUND

Implantable optical modulation systems can provide therapeutic benefits in a variety of diseases and disorders. For example, optical modulation can be applied to the brain either externally or using an implanted modulation lead to provide, for example, deep brain modulation, to treat a variety of diseases or disorders. Optical modulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the nerves, muscles, or other tissue to be stimulated.

### BRIEF SUMMARY

One aspect is an optical modulation system that includes a display; a memory including instructions; a processor coupled to the display and the memory and configured to execute the instructions, wherein the instructions include presenting, on the display, a user interface including a plurality of user-selectable controls to select or adjust values for each of a plurality of modulation parameters; receiving a target; receiving or determining a location of an optical modulation lead relative to the target; presenting, on the display, a representation of the optical modulation lead and a representation of the target relative to the location of the optical modulation lead; estimating a modulation dosage or irradiance at the target based on, at least, the target, the location of the optical modulation lead, the values of the modulation parameters, and estimated light attenuation by tissue between the optical modulation lead and the target; and presenting, on the display, a value of the estimated modulation dosage or irradiance at the target.

Another aspect is a method for optical modulation or programming optical modulation. The method includes presenting, on a display, a user interface including a plurality of user-selectable controls to select or adjust values for each of a plurality of modulation parameters; receiving a target; receiving or determining a location of an optical modulation lead relative to the target; presenting, on the display, a representation of the optical modulation lead and a representation of the target relative to the location of the optical modulation lead; estimating a modulation dosage or irradiance at the target based on, at least, the target, the location of the optical modulation lead, the values of the modulation parameters, and estimated light attenuation by tissue between the optical modulation lead and the target; and presenting, on the display, a value of the estimated modulation dosage or irradiance at the target.

Yet another aspect is a non-transitory computer readable medium have instructions stored thereon, wherein the instructions include presenting, on a display, a user interface including a plurality of user-selectable controls to select or adjust values for each of a plurality of modulation parameters; receiving a target; receiving or determining a location of an optical modulation lead relative to the target; presenting, on the display, a representation of the optical modulation lead and a representation of the target relative to the location of the optical modulation lead; estimating a modulation dosage or irradiance at the target based on, at least, the target, the location of the optical modulation lead, the values of the modulation parameters, and estimated light attenuation by tissue between the optical modulation lead and the target; and presenting, on the display, a value of the estimated modulation dosage or irradiance at the target.

In at least some aspects, the instructions or method further include presenting, on the display, a pointer, and the value of the estimated modulation dosage or irradiance at the site of the pointer relative to the representation of the lead. In at least some aspects, the value of the modulation dosage or irradiance at the target is an instantaneous value of the irradiance at the target. In at least some aspects, the value of the modulation dosage or irradiance at the target is a value of the modulation dosage or irradiance delivered at the target over a defined area. In at least some aspects, the value of the modulation dosage or irradiance at the target is a cumulative value of the modulation dosage delivered at the target for a selected period of time. In at least some aspects, the value of the modulation dosage or irradiance at the target is a value of the modulation dosage delivered at the target for a predefined modulation dosage period.

In at least some aspects, the instructions or method further include presenting, on the display, a representation of an estimated light distribution. In at least some aspects, presenting the representation of the estimated light distribution includes presenting the representation of the estimated light distribution using a plurality of colors, a plurality of shades, or at least one contour line to delineate regions based on a) an estimated amount of irradiance or modulation dosage received in each of the regions or b) an estimated temperature or temperature change for each of the regions. In at least some aspects, the location of the optical modulation lead comprises the location of at least one light emitter of the optical modulation lead.

A further aspect is an optical modulation system that includes a display; a memory including instructions; a processor coupled to the display and the memory and configured to execute the instructions, wherein the instructions include presenting, on the display, a user interface including a plurality of user-selectable controls to select or adjust values for each of a plurality of modulation parameters; receiving a target; receiving or determining a location of an optical modulation lead relative to the target; receiving a selection of the value for each of a plurality of the modulation parameters; and estimating a modulation dosage at the target based on, at least, the target, the location of the optical modulation lead, and the selected values of the modulation parameters, and at least one of a) estimated light attenuation by tissue between the optical modulation lead and the target or b) estimated light distribution with the tissue.

Another aspect is a method for optical modulation or programming optical modulation. The method includes presenting, on a display, a user interface including a plurality of user-selectable controls to select or adjust values for each of a plurality of modulation parameters; receiving a target; receiving or determining a location of an optical modulation lead relative to the target; receiving a selection of the value for each of a plurality of the modulation parameters; and estimating a modulation dosage at the target based on, at least, the target, the location of the optical modulation lead, and the selected values of the modulation parameters, and at least one of a) estimated light attenuation by tissue between the optical modulation lead and the target or b) estimated light distribution with the tissue.

Yet another aspect is a non-transitory computer readable medium having instructions stored thereon, wherein the instructions include presenting, on a display, a user interface including a plurality of user-selectable controls to select or adjust values for each of a plurality of modulation parameters; receiving a target; receiving or determining a location of an optical modulation lead relative to the target; receiving a selection of the value for each of a plurality of the modulation parameters; and estimating a modulation dosage at the target based on, at least, the target, the location of the optical modulation lead, and the selected values of the modulation parameters, and at least one of a) estimated light attenuation by tissue between the optical modulation lead and the target or b) estimated light distribution with the tissue.

In at least some aspects, the instructions or method further include presenting, on the display, a representation of the optical modulation lead and a representation of the target relative to the location of the optical modulation lead. In at least some aspects, the instructions or method further include presenting, on the display, a value of the estimated modulation dosage at the target.

In at least some aspects, the instructions or method further include presenting, on the display, a representation of an estimated light distribution. In at least some aspects, presenting the representation of the estimated light distribution includes presenting the representation of the estimated light distribution using a plurality of colors, a plurality of shades, or at least one contour line to delineate regions based on a) an estimated amount of irradiance or modulation dosage received in each of the regions or b) an estimated temperature or temperature change for each of the regions.

A further aspect is an optical modulation system that includes a display; a memory including instructions; a processor coupled to the display and the memory and configured to execute the instructions, wherein the instructions include presenting, on the display, a user interface including a plurality of user-selectable controls to select or adjust values for each of a plurality of modulation parameters; receiving a target; receiving or determining a location of an optical modulation lead relative to the target; and receiving a selection of the value for each of a plurality of the modulation parameters, wherein the selection of the value of at least one of the modulation parameters is limited by at least one of a) an estimation, by the processor, of a modulation dosage, temperature change, temperature, heating, or delivered energy at the target based on, at least, the target, the location of the optical modulation lead, and the selected value of at least one other of the modulation parameters or b) an estimation, by the processor, of a dosage, temperature change, temperature, heating, or delivered energy at non-target tissue based on, at least, a location of the non-target tissue, a threshold value, the location of the optical modulation lead, and the selected value of at least one other of the modulation parameters.

Another aspect is a method for optical modulation or programming optical modulation. The method includes presenting, on a display, a user interface including a plurality of user-selectable controls to select or adjust values for each of a plurality of modulation parameters; receiving a target; receiving or determining a location of an optical modulation lead relative to the target; and receiving a selection of the value for each of a plurality of the modulation parameters, wherein the selection of the value of at least one of the modulation parameters is limited by at least one of a) an estimation, by the processor, of a modulation dosage, temperature change, temperature, heating, or delivered energy at the target based on, at least, the target, the location of the optical modulation lead, and the selected value of at least one other of the modulation parameters or b) an estimation, by the processor, of a dosage, temperature change, temperature, heating, or delivered energy at non-target tissue based on, at least, a location of the non-target tissue, a threshold value, the location of the optical modulation lead, and the selected value of at least one other of the modulation parameters.

Yet another aspect is a non-transitory computer readable medium having instructions stored thereon, wherein the instructions include presenting, on a display, a user interface including a plurality of user-selectable controls to select or adjust values for each of a plurality of modulation parameters; receiving a target; receiving or determining a location of an optical modulation lead relative to the target; and receiving a selection of the value for each of a plurality of the modulation parameters, wherein the selection of the value of at least one of the modulation parameters is limited by at least one of a) an estimation, by the processor, of a modulation dosage, temperature change, temperature, heating, or delivered energy at the target based on, at least, the target, the location of the optical modulation lead, and the selected value of at least one other of the modulation parameters or b) an estimation, by the processor, of a dosage, temperature change, temperature, heating, or delivered energy at non-target tissue based on, at least, a location of the non-target tissue, a threshold value, the location of the optical modulation lead, and the selected value of at least one other of the modulation parameters.

In at least some aspects, the value of the at least one of the modulation parameters that is limited is the value of the modulation intensity. In at least some aspects, the estimation of the modulation dosage is an estimation of the modulation dosage over a predefined modulation dosage period. In at least some aspects, the estimation of the dosage, temperature change, temperature, heating, or delivered energy at the non-target tissue is an estimation of the dosage, temperature change, temperature, heating, or delivered energy over a predefined period of time. In at least some aspects, the value of the at least one of the modulation parameters is limited when the estimation of the modulation dosage or the estimation of the dosage, temperature change, temperature, heating, or delivered energy exceeds a preselected value.

In at least some aspects, the instructions or method further include presenting, on the display, a representation of an estimated light distribution. In at least some aspects, presenting the representation of the estimated light distribution includes presenting the representation of the estimated light distribution using a plurality of colors, a plurality of shades, or at least one contour line to delineate regions based on an estimated amount of irradiance or modulation dosage received in each of the regions. In at least some aspects, presenting the representation of the estimated light distribution includes presenting the representation of the estimated light distribution using a plurality of colors, a plurality of shades, or at least one contour line to delineate regions based on an estimated temperature or temperature change for each of the regions.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an optical modulation system that includes a lead coupled to a control module;

FIG. 2A is a schematic side view of one embodiment of the control module of FIG. 1 configured and arranged to couple to an elongated device;

FIG. 2B is a schematic side view of one embodiment of a lead extension configured and arranged to couple the elongated device of FIG. 2A to the control module of FIG. 1;

FIG. 3 is a schematic overview of one embodiment of components of an optical modulation system, including an electronic subassembly disposed within a control module;

FIG. 4A is a schematic illustration of one embodiment of a user interface for selecting a target;

FIG. 4B is a schematic illustration of another embodiment of a user interface for selecting a target;

FIG. 5 is a schematic illustration of one embodiment of a user interface for representing the placement of a lead relative to a target;

FIG. 6A is a schematic illustration of one embodiment of a user interface for controlling and estimating an effect of optical modulation; and

FIG. 6B is a schematic illustration of another embodiment of a user interface for controlling and estimating an effect of optical modulation.

### DETAILED DESCRIPTION

The present invention is directed to the area of implantable optical modulation systems and methods of making and using the systems. The present invention is also directed to systems and methods for visualizing and controlling optical modulation using optical modulation leads and systems, as well as user interfaces for optical modulation systems.

In some embodiments, an implantable optical modulation system only provides optical modulation. Examples of optical modulation systems with leads are found in, for example, U.S. Pat. Nos. 9,415,154; 10,335,607; 10,625,072; and 10,814,140 and U.S. Patent Applications Publication Nos. 2020/0155584; 2020/0376272; 2021/0008388; 2021/0008389; 2021/0016111; and 2022/0072329, all of which are incorporated by reference in their entireties.

In other embodiments, the system can provide both optical modulation and electrical stimulation. In at least some of these embodiments, the optical modulation system can be a modification of an electrical stimulation system to also provide optical modulation. Suitable implantable electrical stimulation systems that can be modified to also provide optical modulation include, but are not limited to, a least one lead with one or more electrodes disposed along a distal end of the lead and one or more terminals disposed along the one or more proximal ends of the lead. Examples of electrical stimulation systems with leads are found in, for example, U.S. Pat. Nos. 6,181,969; 6,516,227; 6,609,029; 6,609,032; 6,741,892; 7,203,548; 7,244,150; 7,450,997; 7,596,414; 7,610,103; 7,672,734; 7,761, 165; 7,783,359; 7,792,590; 7,809,435;

7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316;

2012/0203320; 2012/0203321; 2012/0316615; and 2013/0105071; and U.S. patent application Ser. Nos. 12/177,823 and 13/750,725, all of which are incorporated by reference in their entireties.

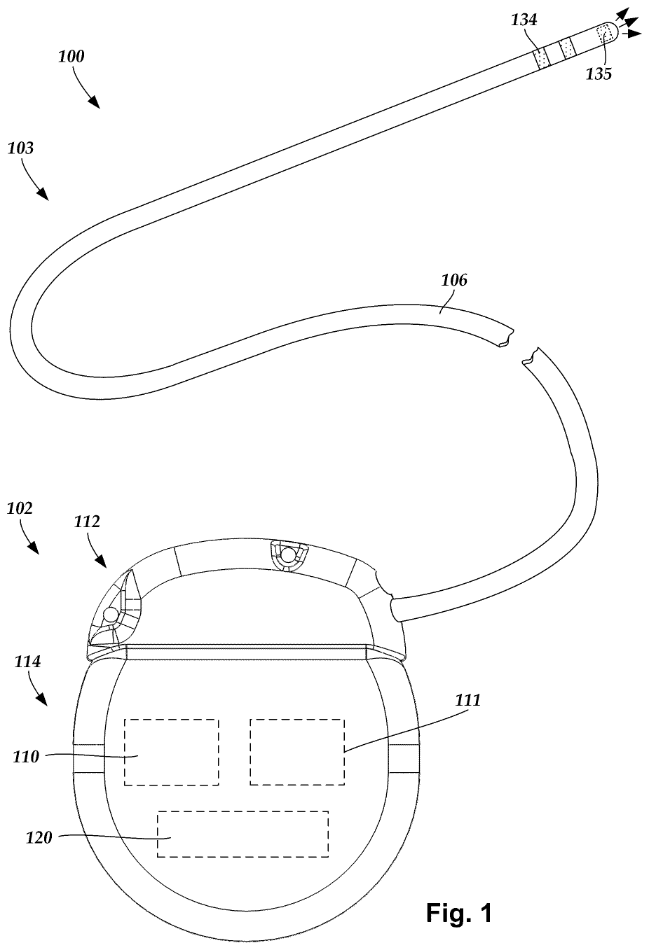

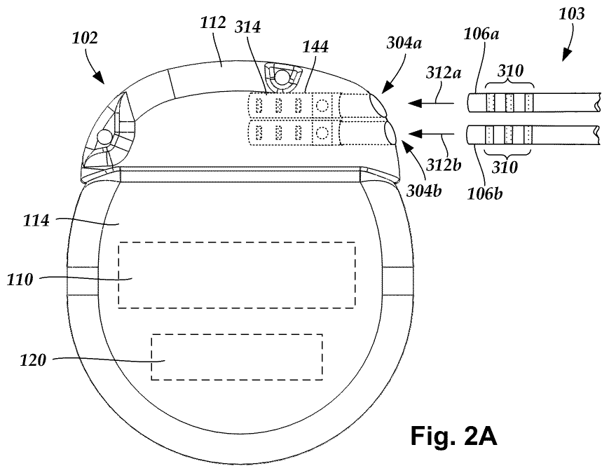

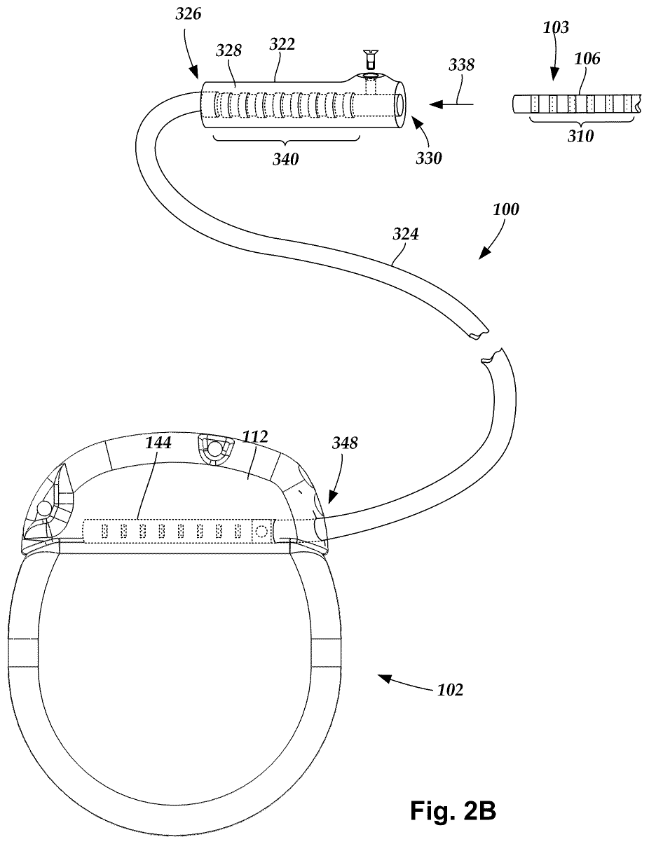

FIG. 1 illustrates schematically one embodiment of an optical modulation system 100. The optical modulation system includes a control module (e.g., a stimulator) 102 and a lead 103 coupleable to the control module 102. The lead 103 includes one or more lead bodies 106. In FIG. 1, the lead 103 is shown having a single lead body 106. In FIG. 2A, the lead 103 includes two lead bodies 106a, 106b. It will be understood that the lead 103 can include any suitable number of lead bodies including, for example, one, two, three, four, five, six, seven, eight or more lead bodies 106.

At least one light emitter 135 is provided along a distal portion of the lead 103. The light emitter 135 can be a light source, such as a light-emitting diode (“LED”), laser diode, organic light-emitting diode (“OLED”), or the like, or can be a terminus of a light transmission element, such as an optical fiber, fiber optic, optical waveguide, or the like, in which case the light source is distant from the distal portion of the lead (for example, in the control module or in a proximal portion of the lead). The lead can also include optional electrodes 134 disposed along the lead body 106, and one or more terminals 310 (FIGS. 2A-2B) disposed along each of the one or more lead bodies 106 and coupled to the electrodes 134 by conductors (not shown). In at least some embodiments, one or more terminals 310 (FIGS. 2A-2B) may also be used to convey electrical signals to a light source that acts as the light emitter 135 by conductors (not shown) extending along the lead.

The optional electrodes 134 can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, iridium, iridium oxide, palladium rhodium, or titanium. In at least some embodiments, at least one of the electrodes 134 is formed from an optically-transparent material. Any suitable number of electrodes 134 can be disposed on the lead including, for example, one, two, three, four, five, six, seven, eight, nine, ten, eleven, twelve, fourteen, sixteen, twenty-four, thirty-two, or more electrodes 134.

The lead 103 can be coupled to the control module 102 in any suitable manner. In some embodiments, the lead is permanently attached to the control module 102. In other embodiments, the lead can be coupled to the control module 102 by a connector 144 (FIG. 2A). In FIG. 2A, the lead 103 is shown coupling directly to the control module 102 through the connector 144. In at least some other embodiments, the lead 103 couples to the control module 102 via one or more intermediate devices, as illustrated in FIG. 2B. For example, in at least some embodiments, one or more lead extensions 324 (FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the optical modulation system 100 includes multiple elongated devices disposed between the lead 103 and the control module 102, the intermediate devices may be configured into any suitable arrangement.

The control module 102 can include, for example, a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

In some embodiments, the control module 102 also includes one or more light sources 111 disposed within the sealed electronics housing 114. In alternate embodiments, the one or more light sources 111 are external to the control module. The one or more light sources can be, for example, a light-emitting diode (“LED”), laser diode, organic light-emitting diode (“OLED”), or the like. When the control module 102 includes multiple light sources, the light sources can provide light in at a same wavelength or wavelength band or some, or all, of the light sources can provide light at different wavelength or different wavelength bands. When the one or more light sources 111 are external to the lead(s), the light emitted by the light sources can be directed to one or more optical fibers, fiber optics, optical waveguides, or the like. The optical fiber, or a series of optical fibers, can transmit the light from the one or more light sources 111 through the control module 102 and lead 103 to the light emitter 135 (which can be terminus of the optical fiber). In at least some embodiments, the optical fiber is a single mode optical fiber. In other embodiments, the optical fiber is a multi-mode optical fiber. In some embodiments, the system includes a single optical fiber. In other embodiments, the system may employ multiple optical fibers in series or in parallel.

In other embodiments, the light emitter 135 can also be the light source, or a combination of light sources, with conductors extending along the lead 103 and coupled to the electronic subassembly 110 to provide signals and power for operating the light source. In yet other embodiments, the light source can be disposed elsewhere in the control module 102, on the lead 103, in another element such as a lead extension, splitter, adaptor, or other stand-alone element.

The optical modulation system or components of the optical modulation system, including the lead 103 and the control module 102, are typically implanted into the body of a patient. The optical modulation system can be used for a variety of applications including, but not limited to brain modulation, deep brain modulation, neural modulation, spinal cord modulation, muscle modulation, sacral nerve modulation, dorsal root ganglion modulation, peripheral nerve modulation, and the like.

The one or more lead bodies 106 are made of a non-conductive, biocompatible material such as, for example, silicone, polyurethane, polyether ether ketone (“PEEK”), epoxy, and the like or combinations thereof. The one or more lead bodies 106 may be formed in the desired shape by any process including, for example, molding (including injection molding), casting, and the like.

One or more terminals 310 (FIGS. 2A-2B) are typically disposed along the proximal end of the one or more lead bodies 106 of the optical modulation system 100 (as well as any splitters, lead extensions, adaptors, or the like) for electrical connection to corresponding connector contacts (e.g., 314 in FIGS. 2A-2B). The connector contacts are disposed in connectors (for example, 144 in FIGS. 1-2B; and 322 FIG. 2B) which, in turn, are disposed on, for example, the control module 102 (or a lead extension, a splitter, an adaptor, or the like). Electrically conductive wires, cables, or the like (not shown) extend from the terminals to the light emitter 135 or electrodes 134.

The electrically-conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal portion of lead 103 or other elongated device configured and arranged for coupling to one embodiment of the control module connector 144. The one or more elongated devices may include, for example, one or more of the lead bodies 106 of FIG. 1, one or more intermediate devices (for example, a splitter, the lead extension 324 of FIG. 2B, an adaptor, or the like or combinations thereof), or any combination thereof.

The control module connector 144 defines at least one port into which a proximal end of the lead 103 or other elongated device can be inserted, as shown by directional arrows 312a and 312b. In FIG. 2A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the lead 103 or other elongated device is inserted into the ports 304a and 304b, the connector contacts 314 can be aligned with a plurality of terminals 310 disposed along the proximal end(s) of the lead(s) 103 or other elongated device(s) to electrically couple the control module 102 to the light emitter 135 or electrodes 134 (FIG. 1) disposed on the lead 103. Each of the terminals 310 can couple to the light emitter 135 or one or more of the electrodes 134. Examples of connectors in control modules are found in, for example, U.S. Pat. Nos. 7,244,150 and 8,224,450, which are incorporated by reference.

FIG. 2B is a schematic side view of another embodiment of the optical modulation system 100. The optical modulation system 100 includes a lead extension 324 that is configured and arranged to couple the control module 102 to one or more leads 103 or other elongated devices, such as a splitter, an adaptor, another lead extension, or the like or any combination thereof. In FIG. 2B, the lead extension 324 is shown coupled to a single port 304 defined in the control module connector 144. Additionally, the lead extension 324 is shown configured and arranged to couple to a single lead 103 or other elongated device. In alternate embodiments, the lead extension 324 is configured and arranged to couple to multiple ports 304 defined in the control module connector 144 (for example, the ports 304a and 304b of FIG. 1), or to receive multiple leads 103 or other elongated devices, or both.

A lead extension connector 322 is disposed on the lead extension 324. In FIG. 2B, the lead extension connector 322 is shown disposed at a distal portion 326 of the lead extension 324. The lead extension connector 322 includes a connector housing 328. The connector housing 328 defines at least one port 330 into which terminals 310 of the lead 103 or other elongated device can be inserted, as shown by directional arrow 338. Each of the terminals 310 can couple to the light emitter 135 or one or more of the electrodes 134. The connector housing 328 also includes a plurality of connector contacts, such as connector contact 340. When the lead 103 or other elongated device is inserted into the port 330, the connector contacts 340 disposed in the connector housing 328 can be aligned with the terminals 310 of the lead 103 or other elongated device to electrically couple the lead extension 324 to the electrodes 134 (FIG. 1) or light emitter 135 (FIG. 1) disposed along the lead 103.

In at least some embodiments, the proximal end of the lead extension 324 is similarly configured and arranged as a proximal end of the lead 103 or other elongated device. The lead extension 324 may include a plurality of electrically-conductive wires (not shown) that electrically couple the connector contacts 340 to a proximal portion 348 of the lead extension 324 that is opposite to the distal portion 326. In at least some embodiments, the conductive wires disposed in the lead extension 324 can be electrically coupled to a plurality of terminals (not shown) disposed along the proximal portion 348 of the lead extension 324. In at least some embodiments, the proximal portion 348 of the lead extension 324 is configured and arranged for insertion into a connector disposed in another lead extension (or another intermediate device). In other embodiments (and as shown in FIG. 2B), the proximal portion 348 of the lead extension 324 is configured and arranged for insertion into the control module connector 144.

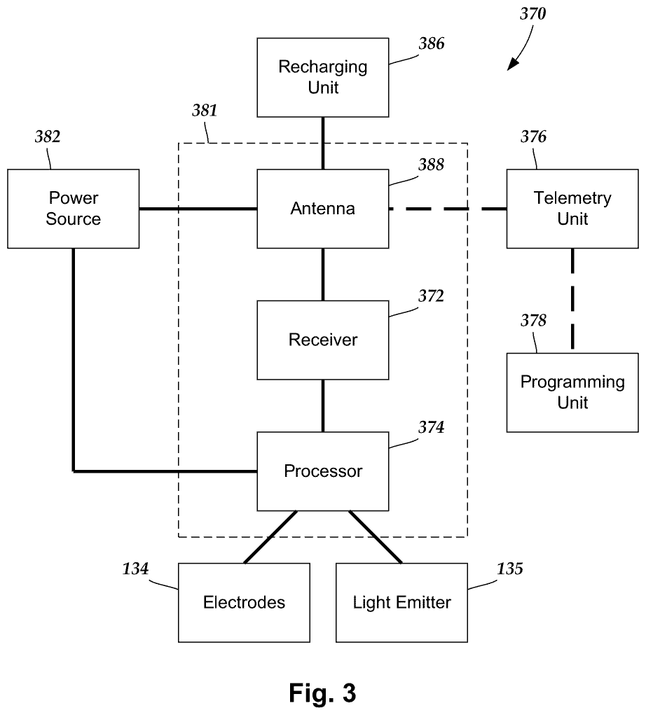

FIG. 3 is a schematic overview of one embodiment of components of an optical modulation system 370 including an electronic subassembly 381 disposed within a control module. It will be understood that the optical modulation system can include more, fewer, or different components and can have a variety of different configurations including those configurations disclosed in the stimulator references cited herein.

Some of the components (for example, a power source 382, an antenna 388, a receiver 372, and a processor 374) of the optical modulation system can be positioned on one or more circuit boards or similar carriers within a sealed housing of an implantable pulse generator, if desired. Any power source 382 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 388 or a secondary antenna. The external power source can be in a device that is mounted on the skin of the user or in a unit that is provided near the user on a permanent or periodic basis.

If the power source 382 is a rechargeable battery, the battery may be recharged using the optional antenna 388, if desired. Power can be provided to the battery for recharging by inductively coupling the battery through the antenna to a recharging unit 386 external to the user. Examples of such arrangements can be found in the references identified above.

In one embodiment, light is emitted by the light emitter 135 of the lead body to stimulate neurons, neuronal cells, non-neuronal cells, nerve fibers, muscle fibers, or other body tissues near the optical modulation system. The processor 374 is generally included to control the timing and other characteristics of the optical modulation system. For example, the processor 374 can, if desired, control one or more of the modulation intensity, wavelength, amplitude, pulse width, pulse frequency, burst frequency, burst duration, cycling (e.g., for repeating intervals of time, determining how long to stimulate and how long to not stimulate), and electrode stimulation configuration (e.g., determining electrode polarity and fractionalization) of the optical modulation.

Additionally, the processor 374 can select which, if not all, of the sensing electrodes are activated. Moreover, the processor 394 can control which types of signals the sensing electrodes detect. In at least some embodiments, the sensing electrodes detect a level of neuronal activation, or neuronal firing rates, or both, received directly from the target modulation location. In other embodiments, the sensing electrodes detect one or more other signals received from the target modulation location in addition to, or in lieu of the level of neuronal activation or neuronal firing rates, such as evoked compound action potentials, local field potentials, multiunit activity, electroencephalograms, electrophysiology, or electroneurograms. In at least some embodiments, one or more of the received signals (e.g., evoked compound action potentials, local field potentials, multiunit activity, electroencephalograms, electrophysiology, electroneurograms, or the like) can be used to indirectly measure the level of neuronal activation, or neuronal firing rates, or both, at the target modulation location. In at least some embodiments, the sensing electrodes or other sensors on, or proximate to, the optical modulation lead can detect or measure temperature or heat.

Optionally, the processor 374 can select one or more stimulation electrodes to provide electrical stimulation, if desired. In some embodiments, the processor 374 selects which of the optional stimulation electrode(s) are cathodes and which electrode(s) are anodes.

Any processor can be used and can be as simple as an electronic device that, for example, produces optical modulation at a regular interval or the processor can be capable of receiving and interpreting instructions from an external programming unit 378 that, for example, allows modification of modulation characteristics. In the illustrated embodiment, the processor 374 is coupled to a receiver 372 which, in turn, is coupled to the optional antenna 388. This allows the processor 374 to receive instructions from an external source to, for example, direct the modulation characteristics and the selection of electrodes, if desired.

In one embodiment, the antenna 388 is capable of receiving signals (e.g., RF signals) from an external telemetry unit 376 which is programmed by the programming unit 378. The programming unit 378 can be external to, or part of, the telemetry unit 376. The telemetry unit 376 can be a device that is worn on the skin of the user or can be carried by the user and can have a form similar to a pager, cellular phone, or remote control, if desired. As another alternative, the telemetry unit 376 may not be worn or carried by the user but may only be available at a home station or at a clinician's office. The programming unit 378 can be any unit that can provide information to the telemetry unit 376 for transmission to the optical modulation system 370. The programming unit 378 can be part of the telemetry unit 376 or can provide signals or information to the telemetry unit 376 via a wireless or wired connection. One example of a suitable programming unit is a computer operated by the user or clinician to send signals to the telemetry unit 376.

The signals sent to the processor 374 via the antenna 388 and the receiver 372 can be used to modify or otherwise direct the operation of the optical modulation system. For example, the signals may be used to modify the modulation characteristics of the optical modulation system such as modifying one or more of modulation duration, pulse frequency, waveform, on or off cycling periods, and modulation intensity. The signals may also direct the optical modulation system 370 to cease operation, to start operation, to start charging the battery, or to stop charging the battery. In other embodiments, the optical modulation system does not include the antenna 388 or receiver 372 and the processor 374 operates as programmed.

Optionally, the optical modulation system 370 may include a transmitter (not shown) coupled to the processor 374 and the antenna 388 for transmitting signals back to the telemetry unit 376 or another unit capable of receiving the signals. For example, the optical modulation system 370 may transmit signals indicating whether the optical modulation system 370 is operating properly or not or indicating when the battery needs to be charged or the level of charge remaining in the battery. The processor 374 may also be capable of transmitting information about the modulation characteristics so that a user or clinician can determine or verify the characteristics.

Conventionally, measurements of optical output of an optical lead are made at the light source or at the site(s) of light emission from the lead. Examples of such measurements are presented in U.S. Patent Applications Publication Nos. 2021/0008388; 2021/0008389; and 2021/0016111, all of which are incorporated by reference in their entireties.

In contrast, as described herein, a system can provide to a user an estimated irradiance or modulation dosage at a target based, in part, on device parameters and the distance of the light emitter(s) from the target. In at least some instances, the estimated irradiance can facilitate more accurate dosing than an estimate of output at the light source or the lead because intervening tissue and other factors can reduce the actual amount of light received at the target. Dosages and parameter selection can be dependent on the target. In at least some embodiments, the modulation dosage can be the amount of energy delivered over a specified, preselected, selected, or predetermined period of time.

It will be recognized that there can be multiple targets. In at least some embodiments, the methods and systems described herein can receive multiple targets and provide information, estimates, or the like accounting for the multiple targets. For example, optical modulation may be directed to two targets such as the substantia nigra pars reticulata (SNr) and the nucleus basalis of Meynert.

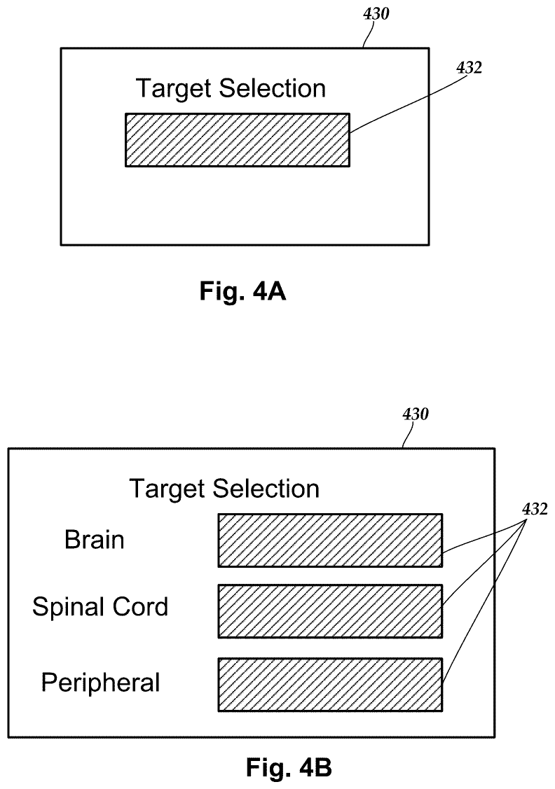

An optical modulation system or a method for optical modulation can include one or more user interfaces for user selection, modification, or alteration of aspects of the optical modulation. For example, a user interface can be target specific or can allow for receiving one or more targets. The receiving of a target can include, but is not limited, to selecting a target or obtaining a target from a device, software, or any other suitable source. For example, FIG. 4A illustrates one embodiment of a user interface 430 for target selection including a selection control 432 for picking a target. The selection control 432 can be a drop-down menu, any other type of menu, a box for typing in the target, importing data that encodes the target (followed by selecting the target), or any other suitable mechanism for selecting a target. For example, a menu of targets can include a list of anatomical regions, such as regions of the brain. FIG. 4B illustrates another user interface 430 which includes a selection control 432 for each of the different types of modulation including, for example, brain modulation, spinal cord modulation, and peripheral (e.g., peripheral nerve) modulation. For example, the selection control 432, when selected, can present a menu of targets for that type of modulation. As an example, for brain modulation, the targets can be regions of the brain such as, for example, the substantia nigra, nucleus basalis of Meynert, or the like. As another example, for the spinal cord modulation, the targets can be different vertebral locations or vertebral structures, such as the dorsal roots, dorsal columns, dorsal horns, dorsal root ganglia, or the like. For peripheral nerve modulation, a menu of different nerves can be presented. It will be recognized that the features (or select features) of any of the user interfaces described herein can be combined with the features (or select features) of any one or more of the other user interfaces described herein.

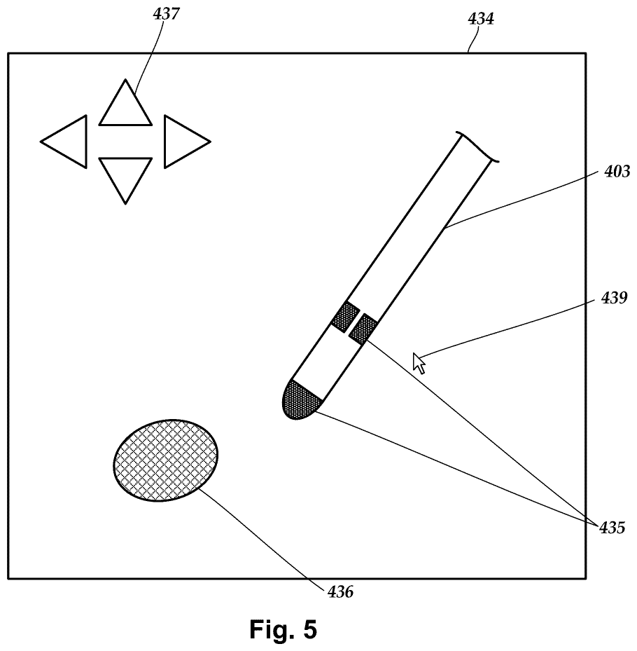

In at least some embodiments, a user interface 434, such as illustrated in FIG. 5, can include the selected target 436 and allow the user to place a lead 403, with one or more light emitters 435, at a position relative to the target based on the user's knowledge or estimation, imaging, or any other information. In at least some embodiments, the user interface 434 can include movement controls 437 for moving the lead 403 within the user interface. In at least some embodiments, the user interface 434 can include a pointer 439 or other mechanism that can be used to move the lead 403 within the user interface (for example, by a drag-and-drop or point-and-click activity.)

Alternatively or additionally, in at least some embodiments, the lead 403 can be placed by the system. Such placement can be based on information provided to the system from the clinician performing the implantation, from any other individual with knowledge or estimation of the lead implantation site, from surgical planning software, from imaging, or the like or any combination thereof.

In at least some embodiments, the user interface 434 presents the lead 403 and target 436 in a two-dimensional plane or a pseudo-three-dimensional arrangement. In at least some embodiments, the lead 403 and target 436 are presented in a plane (or slice) that intersects both the lead and the target.

It will be understood that the light emitter 435 can be a light source or can be an emission region of an optical fiber, fiber optic, or optical waveguide that is coupled to a light source. In at least some embodiments, multiple light sources can be used. In at least some embodiments, a lead can have multiple light sources or light emission regions.

In at least some embodiments, multiple light sources can be used. The multiple light sources can emit light having the same wavelength or wavelength range or two or more of the light sources can differ in wavelength or wavelength range. In at least some embodiments with light sources that differ in wavelength or wavelength range, the user can select the wavelength or wavelength range for the illumination.

In at least some embodiments, there can be two or more leads 403—at least one lead for each hemisphere of the brain or different leads for different medial-lateral or rostral-caudal locations along the spine or different leads for different nerve branches. In at least some embodiments, the user interface 434 (or any other user interface described herein) can show both leads 403 (either in one display area or two display areas) or can be toggled between the two leads.

In at least some embodiments, a user, such as a physician or clinician, can estimate the distance from the light emitter(s) on the lead 403 to the target. In at least some embodiments, information about the target (for example, type, location, branch, or the like) can be used to estimate the distance from the light emitter(s) on the lead to the target. Other information can be used by the system to estimate the distance information provided to the system from a variety of sources including, but not limited to, the clinician performing the implantation, any other individual with knowledge, or estimation, of the lead implantation site, surgical planning software, imaging, or the like or any combination thereof.

In at least some embodiments, anatomical information, imaging, or other information can be used to identify or estimate the tissue (and corresponding tissue properties) that is between the light emitter and the target. In at least some embodiments, the system can utilize a model, equation, or other arrangement for estimating the light distribution within the tissue. Such model, equation, or other arrangement can account for factors such as, for example, the target itself and light attenuation arising from the tissue between the light emitter(s) and the target. Such models can estimate light attenuation arising from one or more of the following: light absorption, scattering, reflection, refraction, or the like or any combination thereof.

In at least some embodiments, an estimated light distribution can be determined using simulations. In at least some embodiments, an estimated light distribution can be determined from a look-up table, an analytical estimation, or other arrangement that is based on modulations conducted previously. In at least some embodiments, an estimated light distribution may be enhanced or updated based on simulations that occur in the background or offline.

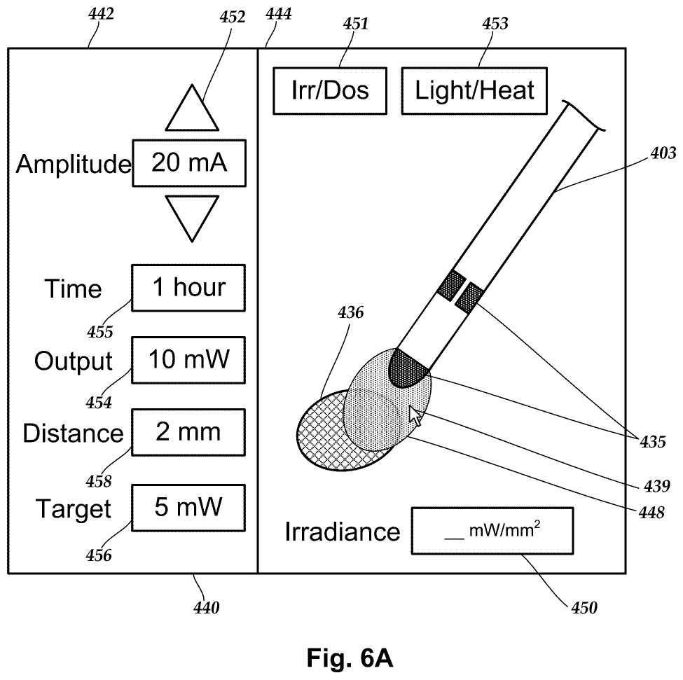

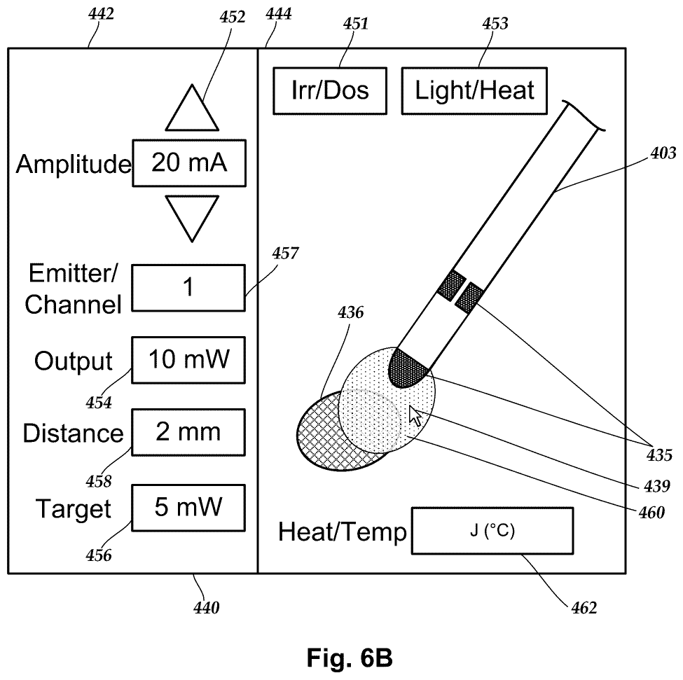

FIG. 6A illustrates one embodiment of a user interface 440 that includes a dose control section 442 and an optional display section 444. In at least some embodiments, the display section 444 includes a representation of the lead 403 and the target 436. The representation of the lead 403 can also include the representation of one or more light emitters 435. In at least some embodiments, the display section 444 can include a pointer 439 that can be moved over the display section by the user.

In at least some embodiments, the display section 444 can include a representation of a light distribution 448 generated by the light emitter(s) 435 according to the current optical modulation parameters. In at least some embodiments, the representation of the light distribution 448 may be limited to, or bounded by, only that area that receives a predetermined or user-selected threshold value of irradiance or modulation dosage. In at least some embodiments, the user can modify the minimum value. In at least some embodiments, the light distribution 448 can be displayed as a pseudo-color, gray-scale, or contour plot. In at least some embodiments, the representation of the estimated light distribution using colors, shades, or contour line(s) to delineate regions based on an estimated amount of irradiance or modulation dosage received in each of the regions. In at least some embodiments, such plots may select color, gray-scale, or contour line(s) that are normalized based on irradiance power. In at least some embodiments, the light distribution is illustrated using a single contour line or a volume that corresponds to irradiance or modulation dosage at or above a threshold amount. In at least some embodiments, a user can set or modify the threshold amount used to determine the single contour line or volume.

In at least some embodiments, the display section 444 may also include a value 450 of the estimated irradiance or modulation dosage at the target 436. This value 450 may be an average value for the target 436, a value at the nearest point of the target to the light emitter(s) 435, a value at a center of the target, or any other suitable value that represents irradiance or modulation dosage at the target. In at least some embodiments, the display section 444 can include any one or more of the following statistical values of irradiance or modulation dosage of the target, such as, for example, a mean, range, or standard deviation. In at least some embodiments, the value 450 of estimated irradiance or modulation dosage may also depend on light source performance over time and determinations of such values may be adjusted during the lifetime of the light source.

In at least some embodiments, the value 450 (or another value display) represents the estimated irradiance or modulation dosage at the point identified by the pointer 439. In at least some embodiments, the system can estimate the value 450 of the irradiance or modulation dosage at the target 436 based on the spot size of the light emitter 435. For example, a spot size estimated to be 1.3 mm may have an irradiance at a target, absent effects arising from intervening tissue, of 4.77 mW/mm².

In at least some embodiments, the user interface 440 can include a control 451 to select or switch between different views of the representation of light distribution 439, value 450, or both. For example, the control 451 be used to can select or switch between the value 450 being irradiance or modulation dosage. As another example, the control 451 can be used to select between a representation of a light distribution 439 (or a value 450 for the modulation dosage) that represents an instantaneous light distribution (e.g., irradiance), a light distribution for a bolus (e.g., a portion of the dosage that constitutes a bolus), a light distribution for the entire modulation dosage (e.g., a daily, weekly, or monthly modulation dosage or other modulation dosage over a defined time period), a light distribution over a defined time period (e.g., a minute, hour, day, week, month, or any other defined period of time), or a cumulative light distribution (where, in at least some embodiments, the start time can be defined by the user or system).

In at least some embodiments, the display section 444 can display estimated heat generation 460 or an estimated temperature or temperature increase in tissue due to the illumination, as illustrated in FIG. 6B. The estimated heat generation, temperature, or temperature increase can be determined and displayed for the target, for intervening or other non-target tissue, or for any combination thereof. Alternatively or additionally, in at least some embodiments, the display section 444 can display estimated heat generation 460 or an estimated temperature or temperature increase in tissue due to the illumination as an overlay around the lead, as a secondary display (for example, as a picture-in-picture arrangement), or using any other suitable display arrangement. In at least some embodiments, the user interface 440 can include a toggle control 453 to toggle between displaying the light distribution 448 or displaying the heat generation 460, temperature, or temperature increase. In at least some embodiments, the estimated heat generation, temperature, or temperature increase is modified based on estimated perfusion or flow of blood which can transport heat away from the site of the estimate.

In at least some embodiments, the display section 444 may also include a value 462 of the estimated heat generation, temperature, or temperature increase at the target 436. This value 462 may be an average value for the target 436, a value at the nearest point of the target to the light emitter(s) 435, a value at a center of the target, or any other suitable value that represents estimated heat generation, temperature, or temperature increase (or any combination thereof) at the target, light emitter, or intervening tissue (or any combination thereof). In at least some embodiments, the display section 444 can include any one or more of the following statistical values of heat generation, temperature, or temperature increase of the target, such as, for example, a mean, range, or standard deviation. In at least some embodiments, the value 462 (or another value display) represents the estimated heat generation, temperature, or temperature increase at the point identified by the pointer 439. In at least some embodiments, a thermal model of the tissue can be used to for estimating heat generation, temperature, or temperature increase.

In FIGS. 6A and 6B, the dose control section 442 can include controls 452 to set or modify parameters such as, for example, amplitude (e.g., amplitude of the current or voltage applied to the light source), modulation intensity, pulse width, pulse rate, burst rate, burst duration, duty cycle, duration of irradiation, wavelength or wavelength range, or the like or any combination thereof. (For ease of illustration, only the control 452 for setting or modifying amplitude is shown.) In at least some embodiments, instead of, or in addition to, setting or modifying an amplitude of the current or voltage applied to the light source, the interface 440 can include controls so that the user can set or modify an output power or output irradiance at the light emitter(s) (control 435) or a desired estimated power or irradiation at the target (control 436). The user interface 440 may also include other information such as the distance 458 between the lead 403 or light emitter(s) 435 and the target 436.

In at least some embodiments, the system may have a preprogrammed default value for one or more of the parameters. In at least some embodiments, the preprogrammed default value(s) may be dependent on the target selected by the user, the type of lead or control module used, or any other suitable conditions or any combination thereof.

In at least some embodiments, the controls 451, 452, 453, 454, 456, 458 can be locked or unlocked by the user, the system, or both. In at least some embodiments, one or more of the parameters can have a preprogrammed upper limit, lower limit, or range. For example, the amplitude or modulation intensity may have an upper limit that can account for one or more of the following: device capabilities, safety, heat generation (with possible damage to tissue or the lead), or the like or any combination thereof.

In at least some embodiments, the selection of a value for at least one of the modulation parameters can be limited by an estimation of a modulation dosage, temperature change, temperature, heating, or delivered energy at the target. This estimation can be based on any number of parameters including, but not limited to, one or more of the target, the location of the optical modulation lead, or the selected value of at least one other of the modulation parameters. In at least some embodiments, the estimation can be defined for a particular period of time (for example, 1, 5, 10, 15, 30, or 90 minutes or 1, 2, 4, 6, or 12 hours, or 1, 2, 5, or 7 days, or the like or any other suitable time period).

In at least some embodiments, the selection of a value for at least one of the modulation parameters can be limited by an estimation of a dosage, temperature change, temperature, heating, or delivered energy at non-target tissue (for example, intervening tissue between the lead 403 and the target 436). This estimation can be based on any number of parameters including, but not limited to, one or more of a location of the non-target tissue, a threshold value, the location of the optical modulation lead, and the selected value of at least one other of the modulation parameters. In at least some embodiments, the estimation can be defined for a particular period of time (for example, 1, 5, 10, 15, 30, or 90 minutes or 1, 2, 4, 6, or 12 hours, or 1, 2, 5, or 7 days, or the like or any other suitable time period).

Examples of systems and methods for estimating dosage, temperature change, temperature, heating, or delivered energy at non-target tissue at target or non-target tissue can be found in U.S. Provisional Patent Application Ser. No. 63/399,982, incorporated herein by reference in its entirety.

In at least some embodiments, the system can include a dosage control (for example, control 456) that limits the delivery of a dosage to a threshold amount. In at least some embodiments, the threshold amount can be user selected or user modified from a default or other value. The threshold amount can correspond to, for example, a level of power delivery, a level of energy delivery, an amount of heat generation at the target or in non-target tissue (for example, intervening tissue), an amount of temperature increase at the target or in non-target tissue, or the like or any combination thereof. In at least some embodiments, the threshold amount can be defined for a particular period of time (for example, 1, 5, 10, 15, 30, or 90 minutes or 1, 2, 4, 6, or 12 hours, or 1, 2, 5, or 7 days, or the like or any other suitable time period). In at least some embodiments, the same controls and considerations can be applied to a bolus of modulation (for example, when the modulation dosage is delivered in two or more boluses) or to continuous modulation.

In at least some embodiments, when the programmed set of parameters is estimated to provide a modulation dosage that exceeds the threshold amount (or generate heat, a temperature, or temperature change above a threshold amount), execution of the set of parameters may be disabled, a warning may be provided to the user, the user interface 440 may display colors or other symbols to indicate that the parameters may be approach or exceed the threshold, or any combination of these actions. For example, parameters may be provided in green, with a green background, or with a green symbol when the generation of light using the parameters will not exceed the threshold amount. Orange can be used when the generation of light using the parameters will likely approach or exceed the threshold amount. Red can be used when the generation of light using the parameters will definitely approach or exceed the threshold amount.

In at least some embodiments, the dose control section 442 also includes an estimated output section which can include information such as the light output 454 (e.g., output power or output irradiance) at the light emitter(s), the distance 448 to the target, and the estimated irradiance or power 456 at the target. The light output 454 is related to parameters such as, amplitude or intensity of light emission (for example, measured in mW), amplitude of the current or voltage that drives the light source, pulse width, and pulse rate. In at least some embodiments, the system estimates the light output 454 based on one or more of these parameters, as explained above.

In the case the that the light emitter(s) 435 is/are not a light source, the system or user may account for losses between the light source(s) and the light emitter(s) when selecting the output power or output irradiance. In at least some embodiments, the output power or output irradiance can be estimated using a transfer function, a calibration table, a calibration formula, or from previous measurement(s). In at least some embodiments, the lead or light source can have an associated light monitor that measures the output power or output irradiance at the light emitter(s) 435 or at the light source. In at least some embodiments, the output power or output irradiance can be expressed as peak irradiant power (mW) or average irradiant power (mW) or any combination thereof. Additional dose information can include the energy per pulse (Joules/pulse).

In at least some embodiments, the power or irradiance at the target can be estimated using a transfer function or from previous measurement(s). In at least some embodiments, power or irradiance at the target can be expressed as peak irradiant power (mW) or average irradiant power (mW) or any combination thereof.

In at least some embodiments, time parameters are reflected in the estimated output power or output irradiance or the estimates are estimates of the energy delivered over a certain period of time. In at least some embodiments, the unit of time is a day, an hour, a minute, or a second, or any other suitable period of time or the unit of time can be a dose (e.g., the amount of irradiance or modulation dosage provided in a day or other suitable time period) or a bolus (e.g., the amount of irradiance or modulation dosage provided as a particular portion of a dose). Time parameters can include, for example, pulse width, pulse rate, duty cycle, selection as continuous wave illumination, duration of a dose or bolus, the number of doses or boluses per day, week, month, or other suitable time period. In at least some embodiments, when a time parameter is set or modified using, for example, a time control 455 (FIG. 6A), the user interface 440 alters the estimated output power, output irradiance, or energy delivered over a certain period of time.

In at least some embodiments, a user can select an energy or modulation dosage (for example, an output 454 at the light emitter or an output 456 at the target) to be delivered (for example, to be delivered per unit of time or to be delivered as a modulation dosage). In at least some embodiments, the system can automatically recommend other parameters, such as amplitude, light emitter selection, pulse width, pulse duration, or the like or any combination thereof, to provide the desired energy.

In at least some embodiments, the user interface 440 can permit the programming of multiple light emitters 435 which can be used simultaneously, sequentially, or in any other arrangement. The dose control section 442 can include a control 457 (FIG. 6B) to select the light emitter(s) for which the current parameters are displayed or available to set or modify.

In at least some embodiments, the user interface 440 can permit the programming of multiple channels (one or more optical modulation channels, one or more electrical stimulation channels, or any combination thereof) or multiple programs (one or more optical modulation programs, one or more electrical stimulation programs, or any combination thereof) which can be used simultaneously, sequentially, or in any other arrangement. In at least some embodiments, controls for optical modulation channels or programs may differ from controls for electrical stimulation channels or programs. The dose control section 442 can include a control 457 (FIG. 6B) to select the channel for which the current parameters are displayed or available to set or modify.

The systems and methods can be used for optogenetic illumination as well. Perfusion of a viral vector or other genetic-modifying agent or photosensitizing agent can facilitate optogenetics therapy or photodynamic therapy. In at least some embodiments, the display section 444 can also display an estimate of perfusion of a viral vector or other genetic-modifying agent or photosensitizing agent. In at least some embodiments, the system can utilize the selected parameters (for example, wavelength selection, illumination parameter selection, and the target selection) to estimate an optogenetic or photodynamic effect.

The methods and systems described herein may be embodied in many different forms and should not be construed as limited to the embodiments set forth herein. Accordingly, the methods and systems described herein may take the form of an entirely hardware embodiment, an entirely software embodiment or an embodiment combining software and hardware aspects. Systems referenced herein typically include memory and typically include methods for communication with other devices including mobile devices. Methods of communication can include both wired and wireless (e.g., RF, optical, or infrared) communications methods and such methods provide another type of computer readable media; namely communication media. Wired communication can include communication over a twisted pair, coaxial cable, fiber optics, wave guides, or the like, or any combination thereof. Wireless communication can include RF, infrared, acoustic, near field communication, Bluetooth™, or the like, or any combination thereof.

It will be understood that each of the methods disclosed herein, can be implemented by computer program instructions. These program instructions may be provided to a processor to produce a machine, such that the instructions, which execute on the processor, create means for implementing the actions. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes, or even in a different sequence than illustrated without departing from the scope or spirit of the invention.

The computer program instructions can be stored on any suitable computer-readable medium including, but not limited to, RAM, ROM, EEPROM, flash memory or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computing device.

The above specification and examples provide a description of the manufacture and use of the invention. Since many embodiments of the invention can be made without departing from the spirit and scope of the invention, the invention also resides in the claims hereinafter appended.

1. An optical modulation system, comprising:
a display;
a memory comprising instructions;
a processor coupled to the display and the memory and configured to execute the instructions, wherein the instructions comprise
presenting, on the display, a user interface comprising a plurality of user-selectable controls to select or adjust values for each of a plurality of modulation parameters;
receiving a target;
receiving or determining a location of an optical modulation lead relative to the target;
presenting, on the display, a representation of the optical modulation lead and a representation of the target relative to the location of the optical modulation lead;
estimating a modulation dosage or irradiance at the target based on, at least, the target, the location of the optical modulation lead, the values of the modulation parameters, and estimated light attenuation by tissue between the optical modulation lead and the target; and
presenting, on the display, a value of the estimated modulation dosage or irradiance at the target.

2. The optical modulation system of claim 1, wherein the instructions further comprise presenting, on the display, a pointer, and the value of the estimated modulation dosage or irradiance at the site of the pointer relative to the representation of the lead.

3. The optical modulation system of claim 1, wherein the value of the modulation dosage or irradiance at the target is an instantaneous value of the irradiance at the target.

4. The optical modulation system of claim 1, wherein the value of the modulation dosage or irradiance at the target is a cumulative value of the modulation dosage delivered at the target for a selected period of time.

5. The optical modulation system of claim 1, wherein the value of the modulation dosage or irradiance at the target is a value of the modulation dosage delivered at the target for a predefined modulation dosage period.

6. The optical modulation system of claim 1, wherein the instructions further comprise presenting, on the display, a representation of an estimated light distribution.

7. The optical modulation system of claim 6, wherein presenting the representation of the estimated light distribution comprises presenting the representation of the estimated light distribution using a plurality of colors, a plurality of shades, or at least one contour line to delineate regions based on a) an estimated amount of irradiance or modulation dosage received in each of the regions or b) an estimated temperature or temperature change for each of the regions.

8. An optical modulation system, comprising:
a display;
a memory comprising instructions;
a processor coupled to the display and the memory and configured to execute the instructions, wherein the instructions comprise
presenting, on the display, a user interface comprising a plurality of user-selectable controls to select or adjust values for each of a plurality of modulation parameters;
receiving a target;
receiving or determining a location of an optical modulation lead relative to the target;
receiving a selection of the value for each of a plurality of the modulation parameters; and
estimating a modulation dosage at the target based on, at least, the target, the location of the optical modulation lead, and the selected values of the modulation parameters, and at least one of a) estimated light attenuation by tissue between the optical modulation lead and the target or b) estimated light distribution with the tissue.

9. The optical modulation system of claim 8, wherein the instructions further comprise presenting, on the display, a representation of the optical modulation lead and a representation of the target relative to the location of the optical modulation lead.

10. The optical modulation system of claim 8, wherein the instructions further comprise presenting, on the display, a value of the estimated modulation dosage at the target.

11. The optical modulation system of claim 8, wherein the instructions further comprise presenting, on the display, a representation of an estimated light distribution.

12. The optical modulation system of claim 11, wherein presenting the representation of the estimated light distribution comprises presenting the representation of the estimated light distribution using a plurality of colors, a plurality of shades, or at least one contour line to delineate regions based on a) an estimated amount of irradiance or modulation dosage received in each of the regions or b) an estimated temperature or temperature change for each of the regions.

13. An optical modulation system, comprising:
a display;
a memory comprising instructions;
a processor coupled to the display and the memory and configured to execute the instructions, wherein the instructions comprise
presenting, on the display, a user interface comprising a plurality of user-selectable controls to select or adjust values for each of a plurality of modulation parameters;
receiving a target;
receiving or determining a location of an optical modulation lead relative to the target; and
receiving a selection of the value for each of a plurality of the modulation parameters, wherein the selection of the value of at least one of the modulation parameters is limited by at least one of a) an estimation, by the processor, of a modulation dosage, temperature change, temperature, heating, or delivered energy at the target based on, at least, the target, the location of the optical modulation lead, and the selected value of at least one other of the modulation parameters or b) an estimation, by the processor, of a dosage, temperature change, temperature, heating, or delivered energy at non-target tissue based on, at least, a location of the non-target tissue, a threshold value, the location of the optical modulation lead, and the selected value of at least one other of the modulation parameters.

14. The optical modulation system of claim 13, wherein the value of the at least one of the modulation parameters that is limited is the value of the modulation intensity.

15. The optical modulation system of claim 13, wherein the estimation of the modulation dosage is an estimation of the modulation dosage over a predefined modulation dosage period.

16. The optical modulation system of claim 13, wherein the estimation of the dosage, temperature change, temperature, heating, or delivered energy at the non-target tissue is an estimation of the dosage, temperature change, temperature, heating, or delivered energy over a predefined period of time.

17. The optical modulation system of claim 13, wherein the value of the at least one of the modulation parameters is limited when the estimation of the modulation dosage or the estimation of the dosage, temperature change, temperature, heating, or delivered energy exceeds a preselected value.

18. The optical modulation system of claim 13, wherein the instructions further comprise presenting, on the display, a representation of an estimated light distribution.

19. The optical modulation system of claim 18, wherein presenting the representation of the estimated light distribution comprises presenting the representation of the estimated light distribution using a plurality of colors, a plurality of shades, or at least one contour line to delineate regions based on an estimated amount of irradiance or modulation dosage received in each of the regions.

20. The optical modulation system of claim 18, wherein presenting the representation of the estimated light distribution comprises presenting the representation of the estimated light distribution using a plurality of colors, a plurality of shades, or at least one contour line to delineate regions based on an estimated temperature or temperature change for each of the regions.
